(19) United States
(12) Patent Application Publication
(10) Pub. No.: US 2005/0272138 A1
(43) Pub. Date: Dec. 8, 2005

(54) Method of preparing cross-linked enzyme particles

(75) Inventors: Cesar Mateo, Delft (NL); Lukas Michael van Langen, Delft (NL); Frederik Van Rantwijk, Delft (NL)

Correspondence Address: PEACOCK MYERS, P.C., P O BOX 26927, ALBUQUERQUE, NM, 87125-6927, US

(73) Assignee: Technische Universiteit Delft, Delft (NL)

(21) Appl. No.: 11/125,592

(22) Filed: May 9, 2005

Related U.S. Application Data
(63) Continuation of application No. PCT/NL03/00784, filed on Nov. 7, 2003.

(30) Foreign Application Priority Data
Nov. 8, 2002 (NL) 1021879

(51) Int. Cl. C12N 9/00

(52) U.S. Cl. 435/183

(57) ABSTRACT

The invention relates to a method of preparing cross-linked enzyme particles using a cross-linking agent. According to the invention, the enzyme particles are formed and subsequently cross-linked using a cross-linking agent having at least n reactive groups where N≧3 and a molecular weight of >2,000 Da. The method according to the invention allows for obtaining enzyme particles having a higher enzyme activity than enzyme particles cross-linked according to the state of the art.

The present invention relates to a method of preparing cross-linked enzyme particles using a cross-linking agent.

Such a method is generally known in the art. Often, use is made of glutardialdehyde as a cost-effective and readily available cross-linking agent. The enzyme particles may be enzyme crystals or an enzyme aggregate. Preparing enzyme crystals and aggregates is well known in the art.

Often it is found that the activity of the resulting immobilised enzyme is strongly reduced with respect to the original non-cross-linked enzyme particles.

The object of the present invention is to provide a method which, at least for some enzymes, results in cross-linked enzyme particles having a less reduced enzymatic activity.

The method according to the present invention is characterized in that enzyme particles are formed and subsequently cross-linked using a cross-linking agent having at least n reactive groups where n≧3 and a molecular weight of >2,000 Da.

Applicant has found that for several enzymes cross-linked enzyme particles could be prepared having an activity much higher than that of enzyme particles cross-linked using conventional methods. Advantageously, n is larger than 3, for example >0.5 per 1,000 Da, with a molecular weight of at least 10,000.

Preferably, the cross-linking agent is a water-soluble cross-linking agent having a solubility of at least 0.1 mg/ml.

This allows the method to be performed in an aqueous environment, much appreciated by most enzymes.

Preferably, the cross-linking agent has a molecular weight of at least 5,000 preferably at least 10,000 more preferably at least 25,000.

Without wishing to be bound to any particular theory, it is believed that a higher molecular weight cross-linking agent has difficulty entering enzyme particles and will not react with and inactivate enzyme molecules or the active sites thereof within the enzyme particles.

According to an advantageous embodiment, the cross-linking agent is a polyaldehyde.

A polyaldehyde appeared to be cost-effective and effective cross-linking agent.

According to a preferred embodiment a polysaccharide is subjected to a chemical or enzymatical treatment to yield the polyaldehyde which is subsequently contacted with the enzyme particles to be cross-linked.

Polysaccharides are available at low cost, optionally at a high purity, and can be easily converted into a polyaldehyde.

The polysaccharide is preferably chosen from the group consisting of starch, glycogen and dextrans.

The preferred methods of preparing the polysaccharide involve a) chemical treatment of the polysaccharide with periodate; and b) the enzymatical treatment with a galactose oxidase in the presence of molecular oxygen.

If desired, the bond formed by reaction of an aldehyde group with an amino group of the enzyme is reduced by subjecting the cross-linked enzyme particles to treatment with a reducing agent.

Advantageously, the reducing agent is chosen from the group consisting of sodium borohydride and sodium cyanoborohydride.

The present invention will now be illustrated with reference to the following example:

Activity Assays of the Enzymes:

Nitrilase from Pseudomonas fluorescens:

To 1800 μl of 20 mM phosphate buffer at pH 7.4 was added 100 μl of mandelonitrile solution (13.6 mg in 10 ml of methanol) and 100 μl nitrilase-containing cell-free extract of Pseudomonas fluorescens solution. This reaction was left at 30° C. under stirring. At different times samples of 100 μl were taken and the reaction stopped by addition of 400 μl of an aqueous solution composed of 20% acetonitrile, and 50 mM phosphate buffer at pH 2.2. After stopping the reaction, the quantity of product formed was determined by HPLC. For these assays a Chromolith RP-18e 50-4.6 column (Merck) was used and the mobile phase was 20% acetonitrile, 80% water and 0.1% trifluoroacetic acid.

Alcohol Dehydrogenase from Lactobacillus brevis:

The activity of alcohol dehydrogenase (Jülich Fine Chemicals, Jülich, Germany) was followed spectrophotometrically by the decrease in the absorbance at 340 nm and 30° C. under stirring. The reaction mixture consisted of: 1.940 ml of 11 mM acetophenone and 1 mM MgCl₂ in 50 mM TEA buffer pH 7, 40 μl of 9.5 mM NADPH (in buffer pH 7) and 20 μl diluted enzyme solution.

Oxidation of Dextran:

1.65 g of dextran (MW 100-200 kDa) was dissolved in 50 ml of water and 3.85 g of sodium periodate was added. The final solution was left under stirring at room temperature during 90 minutes. After this time the solution was dialyzed against more than 500 volumes of water.

Preparation of CLEAs:

Using glutardialdehyde (control): To 10 ml of different enzymatic solutions was added 2 ml of 0.5 M phosphate buffer pH 7, 15 ml dimethoxyethane (DME) and 1.6 ml of an aqueous 25% (w/v) glutardialdehyde. The resulting suspension was left under stirring during 16 hours at 4° C. during which period cross-linking takes place. After this time the CLEA was washed with water and dried under vacuum.

Using polyaldehyde dextran (according to the invention): To 1 ml of different enzymatic solutions was added: 1 ml of 500 mM of phosphate buffer at pH 8, 2 ml of oxidated dextran prepared as described above and 4 ml of dimethoxyethane (DME). The resulting suspension was stirred at 4° C. during 16 hours during which period cross-linking takes place. After this time, the obtained CLEAs were resuspended in 40 ml of an aqueous sodium bicarbonate solution (pH=8.5) containing 1 mg/ml of sodium borohydride. This solution was left reacting during 1 hour at 4° C. Finally the reduced CLEAs were washed at 4° C. with water.

Results:

CLEAs with Nitrilase:

When glutardialdehyde was used as crosslinker agent, the final activity obtained was negligible. It is hypothesized that this may be due to a reactive nucleophile at the active site of the enzyme. On the other hand, when polyaldehyde dextran was used as the cross-linking agent, the final activity obtained for the CLEAs was around 50% with respect to the soluble enzyme. Without wishing to be bound by any particular theory, it is believed that the steric hindrance produced by polysaccharide-based cross-linking agents may result in reduced loss of enzymatic activity because of a reduced possibility of reaction between the polymer with the active site.

CLEAs with Alcohol Dehydrogenase:

A complete loss of activity was observed when the enzyme was cross-linked with glutardialdehyde. However, by using polyaldehyde dextrans as the cross-linking agent, it is possible to maintain about 10% of the activity with respect to the soluble enzyme.

While not recommended, it may be possible to add the cross-linking agent to solubilized enzyme before the enzyme aggregate is formed by adding a precipitating agent. However, in accordance with the present invention this is generally not recommended, as this may lead to inactivation of the enzyme. According to the invention, it is preferred that less than 75%, preferably less than 50% and more preferably less than 20% inactivation occurs due to cross-linking before enzyme aggregate formation.

The size of the enzyme particles (aggregates or crystals) is generally between 1 and 50 micron. The particles according to the invention consist mainly of the enzyme material (>75% by wt., more preferably >90%), in contrast to carrier-bound enzymes, which generally contain much less enzyme material, such as less than <10%.

1. A method of preparing cross-linked enzyme particles using a cross-linking agent, comprising:
forming a suspension of enzyme particles; and
cross-linking the enzyme particles using a dissolved cross-linking agent having at least n reactive groups where n is equal to or greater than 3 and wherein the cross-linking agent has a molecular weight of greater than 2,000 Daltons.

2. The method according to claim 1, wherein the cross-linking agent is a water-soluble cross-linking agent having a solubility of at least 0.1 mg/ml.

3. The method according to claim 1, wherein the cross-linking agent has a molecular weight of at least 5,000.

4. The method according to claim 3, wherein the cross-linking agent has a molecular weight of at least 10,000.

5. The method according to claim 3, wherein the cross-linking agent has a molecular weight of at least 25,000.

6. The method according to claim 1, wherein the cross-linking agent is a polyaldehyde.

7. The method according to claim 6, further comprising the step of subjecting a polysaccharide to a chemical or enzymatical treatment to yield the polyaldehyde, which polyaldehyde is subsequently contacted with the enzyme particles to be cross-linked.

8. The method according to claim 7, wherein the polysaccharide is chosen from the group consisting of starch, glycogen and dextrans.

9. The method according to claim 7, wherein the chemical treatment of the polysaccharide comprises treatment with periodate.

10. The method according to claim 8, wherein the enzymatical treatment of the polysaccharide comprises treatment with a galactose oxydase in the presence of molecular oxygen.

11. The method according to claim 6, wherein the cross-linked enzyme particles are subjected to treatment with a reducing agent prior to cross-linking.

12. The method according to claim 11, wherein the reducing agent is chosen from the group consisting of sodium borohydride and sodium cyanoborohydride.
